(19) United States
(12) Patent Application Publication
(10) Pub. No.: US 2008/0003236 A1
(43) Pub. Date: Jan. 3, 2008

(54) ADENOVIRUS FIBER SHAFT COMPOSITION AND METHODS OF USE

(75) Inventors: C. Richter King, Washington, DC (US); Jason G. D. Gall, Germantown, MD (US); Gary J. Nabel, Washington, DC (US); Cheng Cheng, Bethesda, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: GenVec, Inc., Gaithersburg, MD (US); Government of the U.S.A., represented by the Secretary, Department of Health and Human Services, Rockville, MD (US)

(21) Appl. No.: 11/734,851

(22) Filed: Apr. 13, 2007

Related U.S. Application Data
(63) Continuation of application No. PCT/US05/37155, filed on Oct. 17, 2005.
(60) Provisional application No. 60/619,912, filed on Oct. 18, 2004.

(51) Int. Cl. A61K 38/00; A61K 39/00; A61K 39/385; A61P 43/00; C12N 15/00; C12P 21/06

(52) U.S. Cl. 424/192.1; 424/196.11; 435/320.1; 435/69.1; 514/44

(57) ABSTRACT

The invention provides a gene transfer vector and a conjugate comprising at least three contiguous amino acids of a shaft region of a subgroup C adenovirus fiber protein. The invention also provides methods of using the gene transfer vector and the conjugate to induce an immune response in a mammal, and to deliver a protein or a non-proteinaceous molecule to a specific cell type.

### CROSS-REFERENCE TO RELATED APPLICATIONS

This patent application is a continuation of copending International Patent Application No. PCT/US2005/037155, filed Oct. 17, 2005, which claims the benefit of U.S. Provisional Patent Application No. 60/619,912, filed Oct. 18, 2004.

### STATEMENT REGARDING FEDERALLY SPONSORED RESEARCH AND DEVELOPMENT

This invention was made in part with Government support under Cooperative Research and Development Agreement (CRADA) Number AI-1034, and amendments thereto, executed between GenVec, Inc. and the U.S. Public Health Service representing the National Institute of Allergy and Infectious Diseases. The Government may have certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 823 Byte ASCII (Text) file named “701534_ST25.txt,” created on Apr. 7, 2007.

### FIELD OF THE INVENTION

This invention pertains to compositions comprising a portion of an adenovirus fiber protein, and methods of using same.

### BACKGROUND OF THE INVENTION

Delivery of proteins as therapeutics or for inducing an immune response in biologically relevant amounts has been an obstacle to drug and vaccine development for decades. One solution that has proven to be a successful alternative to traditional drug delivery approaches is delivery of exogenous nucleic acid sequences for production of therapeutic factors in vivo. Gene transfer vectors ideally enter a wide variety of cell types, have the capacity to accept large nucleic acid sequences, are safe, and can be produced in quantities required for treating patients. Viral vectors have these advantageous properties and are used in a variety of protocols to treat or prevent biological disorders.

Despite their advantageous properties, widespread use of viral gene transfer vectors is hindered by several factors. In this regard, certain cells are not readily amenable to gene delivery by currently available viral vectors. For example, lymphocytes are impaired in the uptake of adenoviruses (Silver et al., Virology 165, 377-387 (1988); Horvath et al., J. Virology, 62(1), 341-345 (1988)).

The use of viral gene transfer vectors also is impeded by the immunogenicity of viral vectors. A majority of the U.S. population has been exposed to wild-type forms of many of the viruses currently under development as gene transfer vectors (e.g., adenovirus). As a result, much of the U.S. population has developed pre-existing immunity to certain virus-based gene transfer vectors. As a result, such vectors are quickly cleared from the bloodstream, thereby reducing the effectiveness of the vector in delivering biologically relevant amounts of a gene product. Moreover, the immunogenicity of certain viral vectors prevents efficient repeat dosing, which can be advantageous for “boosting” the immune system against pathogens, and results in only a small fraction of a dose of the viral vector delivering its payload to host cells.

Thus, there remains a need for improved methods of delivering therapeutic or antigenic genes and proteins to target cells. The invention provides such a method. These and other advantages of the invention, as well as additional inventive features, will be apparent from the description of the invention provided herein.

### BRIEF SUMMARY OF THE INVENTION

The invention provides a method of inducing an immune response in a mammal comprising administering to the mammal a gene transfer vector comprising (a) a nucleic acid sequence encoding at least one antigen which is expressed in the mammal to induce an immune response, and (b) an amino acid sequence comprising at least three contiguous amino acids of a shaft region of a subgroup C adenovirus fiber protein, wherein the gene transfer vector is not an adenoviral vector.

The invention also provides a method of inducing an immune response in a mammal comprising administering to the mammal a conjugate comprising (a) at least one antigen which induces an immune response in the mammal, and (b) an amino acid sequence comprising at least three contiguous amino acids of a shaft region of a subgroup C adenovirus fiber protein, wherein the conjugate is not an adenovirus.

The invention additionally provides a gene transfer vector comprising (a) a nucleic acid sequence encoding a protein, and (b) an amino acid sequence comprising at least three contiguous amino acids of a shaft region of a subgroup C adenovirus fiber protein, wherein the gene transfer vector is not an adenoviral vector.

The invention further provides a conjugate comprising (a) a protein or a non-proteinaceous molecule, and (b) an amino acid sequence comprising at least three contiguous amino acids of a shaft region of a subgroup C adenovirus fiber protein, wherein when the conjugate comprises a protein, the conjugate is not an adenovirus.

### BRIEF DESCRIPTION OF THE DRAWINGS

FIG. 1A is a graph illustrating the CD4+ T cell response in mice administered with adenoviral vector constructs encoding a green fluorescent protein (GFP).

FIG. 1B is a graph illustrating the CD8+ T cell response in mice administered with adenoviral vector constructs encoding a green fluorescent protein.

FIG. 2 is a graph illustrating luciferase expression following administration of the adenoviral vectors AdlucDA, Adluc, and Adf to murine bone marrow dendritic cells in the presence or absence of a glycosaminoglycan competitor.

FIGS. 3A-3D are each a graph illustrating luciferase expression following administration of the adenoviral vectors AdL and AdDA to 293-ORF6 cells, Caov3 human ovarian adenocarcinoma cells, LL2 murine lung carcinoma cells, and CT26 murine colon carcinoma cells, respectively.

### DETAILED DESCRIPTION OF THE INVENTION

The invention is predicated, at least in part, on the discovery that a subgroup C adenoviral vector ablated for native host cell binding is capable of inducing an immune response in a human host when the adenoviral vector encodes an antigenic transgene. Such an adenoviral vector typically comprises a fiber protein in which the knob domain of the fiber is altered or deleted, but retains the shaft region of the fiber.

The invention provides a method of inducing an immune response in a mammal, which method comprises administering to the mammal a gene transfer vector comprising (a) a nucleic acid sequence encoding at least one antigen which is expressed in the mammal to induce an immune response, and (b) an amino acid sequence comprising at least three contiguous amino acids of a shaft region of a subgroup C adenovirus fiber protein, wherein the gene transfer vector is not an adenoviral vector.

The gene transfer vector can be any suitable gene transfer vector. Examples of suitable gene transfer vectors include plasmids, liposomes, molecular conjugates (e.g., transferrin), and viruses. Preferably, the gene transfer vector is a viral vector. Any suitable viral vector can be used in the inventive method, so long as the viral vector is not an adenoviral vector. Suitable viral vectors include, for example, retroviral vectors, herpes simplex virus (HSV)-based vectors, poxvirus vectors, lentivirus vectors, parvovirus-based vectors, e.g., adeno-associated virus (AAV)-based vectors, and bacteriophage vectors. These viral vectors can be prepared using standard recombinant DNA techniques described in, for example, Sambrook et al., Molecular Cloning, A Laboratory Manual, 3^{rd} edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. As such, long-term expression of a therapeutic factor(s) is achievable when using retrovirus. Retroviruses contemplated for use in gene therapy are relatively non-pathogenic, although pathogenic retroviruses exist. When employing pathogenic retroviruses, e.g., human immunodeficiency virus (HIV) or human T-cell lymphotrophic viruses (HTLV), care must be taken in altering the viral genome to eliminate toxicity to the host. A retroviral vector additionally can be manipulated to render the virus replication-deficient. As such, retroviral vectors are considered particularly useful for stable gene transfer in vivo. Lentiviral vectors, such as HIV-based vectors, are exemplary of retroviral vectors used for gene delivery. Unlike other retroviruses, HIV-based vectors are known to incorporate their passenger genes into non-dividing cells and, therefore, can be of use in treating persistent forms of disease.

An HSV-based viral vector is suitable for use as a gene transfer vector to introduce a nucleic acid into numerous cell types. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the HSV vector are its ability to enter a latent stage that can result in long-term DNA expression, and its large viral DNA genome that can accommodate exogenous DNA inserts of up to 25 kb. Of course, the ability of HSV to promote long-term production of exogenous protein is potentially disadvantageous in terms of short-term treatment regimens. However, one of ordinary skill in the art has the requisite understanding to determine the appropriate vector for a particular situation. HSV-based vectors are described in, for example, U.S. Pat. Nos. 5,837,532, 5,846,782, 5,849,572, and 5,804,413, and International Patent Application Publication Nos. WO 91/02788, WO 96/04394, WO 98/15637, and WO 99/06583.

Because they are highly immunogenic and readily engineered, poxvirus vectors have been used extensively as vaccines against infectious organisms and, more recently, as tumor vaccines. Vaccinia virus and other members of the poxviridae family remain in the cytoplasm and use virally encoded polymerases to carry out replication and transcription. Thus, recombination of viral DNA into the genome is not a concern with vaccinia virus, as it is with other vectors, particularly retroviruses. The infectious cycle is divided into three phases. Early-phase genes, typically encoding proteins with enzymatic function, are expressed before replication. The expression of a small number of intermediate genes depends on replication of the genome and, in turn, drives expression of structural proteins and other products of the late genes.

AAV vectors are viral vectors of particular interest for use in vaccine protocols. AAV is a DNA virus, which is not known to cause human disease. The AAV genome is comprised of two genes, rep and cap, flanked by inverted terminal repeats (ITRs), which contain recognition signals for DNA replication and packaging of the virus. AAV requires co-infection with a helper virus (i.e., an adenovirus or a herpes simplex virus), or expression of helper genes, for efficient replication. AAV can be propagated in a wide array of host cells including human, simian, and rodent cells, depending on the helper virus employed. An AAV vector used for administration of a nucleic acid sequence typically has approximately 96% of the parental genome deleted, such that only the ITRs remain. The use of AAV as a vaccine construct against HIV is reviewed in, for example, Expert. Rev. Vaccines, 1, 7 (2002).

Bacteriophage (or phage) are viruses that grow in bacterial cells. Depending on the complexity of the bacteriophage genome, some bacteriophage rely entirely upon the protein machinery of the host cell for propagation. Bacteriophage propagate by way of a lytic or lysogenic life cycle. A bacteriophage in the lytic cycle converts an infected cell into a “phage factory,” and produces many phage progeny. Bacteriophage capable only of lytic growth often are referred to as a “virulent” bacteriophage. The lysogenic life cycle has been observed only with bacteriophage containing a double-strand DNA genome, and typically involves the bacteriophage integrating into the bacterial chromosome. No progeny bacteriophage particles are produced during the lysogenic cycle. Bacteriophage capable of a lysogenic life cycle are often referred to as “temperate” bacteriophage, and can undergo a lytic cycle under certain conditions, such as DNA damage (see, e.g., Maloy et al., eds., Microbial Genetics, 2^{nd} ed., Jones and Bartlett Publishers, Boston (1994)). Bacteriophage have been modified to specifically target and transduce mammalian cells, (see, e.g., Poul et al., J. Mol. Biol., 288, 203-11 (1999), Monaci et al., Curr. Opin. Mol. Ther., 3, 159-69 (2001), and Larocca et al., Curr. Pharm. Biotechnol., 3, 45-57 (2002)). Bacteriophage suitable for use as gene transfer vectors include, for example, bacteriophage λ and bacteriophage M13. The use of bacteriophage as a human vaccine vector is reviewed in, for example, Clark et al., Expert. Rev. Vaccines, 3, 463-76 (2004).

A virus-like particle is a defective virion which is incapable of infecting a host cell due to the presence of one or more genetic modifications of viral genes or other genetic elements which are functionally critical at some stage of the virus lifecycle. Virus-like particles may or may not contain all of the viral proteins normally found in infectious virions and may or may not contain nucleic acid (i.e., DNA or RNA). If nucleic acid is contained within the particle, it will be incapable of infecting a host cell. Examples of virus like particle vaccine constructs are disclosed in, for example, Schreckenberger et al., Curr. Opin. Oncol., 16, 485-91 (2004).

The gene transfer vector of the inventive method comprises a nucleic acid sequence encoding an antigen which is expressed in the mammal to induce an immune response. An “antigen” is a molecule that triggers an immune response in a mammal. An “immune response” can entail, for example, antibody production and/or the activation of immune effector cells. An antigen in the context of the invention can comprise any subunit of any proteinaceous molecule, including a protein, peptide, or glycoprotein of viral, bacterial, parasitic, fungal, protozoan, prion, cellular, or extracellular origin, which ideally provokes an immune response in mammal, preferably leading to protective immunity. The antigen also can be a self antigen, i.e., an autologous protein which the body reacts to as if it is a foreign invader. The nucleic acid sequence encoding the antigen is not limited to a type of nucleic acid sequence or any particular origin. For example, the nucleic acid sequence can be recombinant DNA, can be genomic DNA, can be obtained from a DNA library of potential antigenic epitopes, or can be synthetically generated.

In one embodiment, the antigen is a viral antigen. The viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae (e.g., Hepatitis B virus), Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B), Papovaviridae, Paramyxoviridae (e.g., measles, mumps, and human respiratory syncytial virus), Parvoviridae, Picornaviridae (e.g., enterovirus, poliovirus, rhinovirus, hepatovirus, and aphthovirus), Plasmodiidae (e.g., Plasmodium falciparum, Plasmodium vivax, Plasmodium ovale, and Plasmodium malariae), Poxviridae (e.g., vaccinia virus), Reoviridae (e.g., rotavirus), Retroviridae (e.g., lentivirus, such as human immunodeficiency virus (HIV) 1 and HIV 2), Rhabdoviridae, and Totiviridae. Preferably, at least one antigen of the inventive method is a retroviral antigen. The retroviral antigen can be, for example, an HIV antigen, such as all or part of the gag, env, or pol protein. Any clade of HIV is appropriate for antigen selection, including clades A, B, C, MN, and the like. Also preferably, at least one antigen encoded by the gene transfer vector is a coronavirus antigen, such as a SARS virus antigen. Suitable SARS virus antigens for the inventive method include, for example, all or part of the E protein, the M protein, and the spike protein of the SARS virus. Suitable viral antigens also include all or part of Dengue protein M, Dengue protein E, Dengue D1NS1, Dengue D1NS2, and Dengue D1NS3. The antigenic peptides specifically recited herein are merely exemplary as any viral protein can be used in the context of the invention.

The antigen can be a parasite antigen such as, but not limited to, a Sporozoan antigen. For example, the nucleic acid sequence can encode a Plasmodian antigen, such as all or part of a Circumsporozoite protein, a Sporozoite surface protein, a liver stage antigen, an apical membrane associated protein, or a Merozoite surface protein.

Alternatively or in addition, at least one antigen encoded by the viral vector is a bacterial antigen. The antigen can originate from any bacterium including, but not limited to, Actinomyces, Anabaena, Bacillus, Bacteroides, Bdellovibrio, Caulobacter, Chlamydia, Chlorobium, Chromatium, Clostridium, Cytophaga, Deinococcus, Escherichia, Halobacterium, Heliobacter, Hyphomicrobium, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema. In a preferred embodiment, at least one antigen encoded by the nucleic acid sequence is a Pseudomonas antigen or a Heliobacter antigen.

It will be appreciated that an entire, intact viral or bacterial protein is not required to produce an immune response. Indeed, most antigenic epitopes are relatively small in size and, therefore, protein fragments can be sufficient for exposure to the immune system of the mammal. In addition, a fusion protein can be generated between two or more antigenic epitopes of one or more antigens. For example, all or part of HIV envelope, e.g., all or part of gp120 or gp160, can be fused to all or part of the HIV pol protein to generate a more complete immune response against the HIV pathogen compared to that generated by a single epitope. Delivery of fusion proteins via a gene transfer vector to a mammal allows exposure of an immune system to multiple antigens and, accordingly, enables a single vaccine composition to provide immunity against multiple pathogens or multiple epitopes of a single pathogen.

Preferably, the nucleic acid is operably linked to (i.e., under the transcriptional control of) one or more promoter and/or enhancer elements, for example, as part of a promoter-variable expression cassette. Techniques for operably linking sequences together are well known in the art. A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Any promoter (i.e., whether isolated from nature or produced by recombinant DNA or synthetic techniques) can be used in connection with the invention to provide for transcription of the nucleic acid sequence. The promoter preferably is capable of directing transcription in a eukaryotic (desirably mammalian) cell. The functioning of the promoter can be altered by the presence of one or more enhancers and/or silencers present on the vector. “Enhancers” are cis-acting elements of DNA that stimulate or inhibit transcription of adjacent genes. An enhancer that inhibits transcription also is termed a “silencer.” Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

Promoter regions can vary in length and sequence and can further encompass one or more DNA binding sites for sequence-specific DNA binding proteins and/or an enhancer or silencer. Enhancers and/or silencers can similarly be present on a nucleic acid sequence outside of the promoter per se. Desirably, a cellular or viral enhancer, such as the cytomegalovirus (CMV) immediate-early enhancer, is positioned in the proximity of the promoter to enhance promoter activity.

Any suitable promoter or enhancer sequence can be used in the context of the invention. In this respect, the antigen-encoding nucleic acid sequence can be operably linked to a viral promoter. Suitable viral promoters include, for instance, cytomegalovirus (CMV) promoters, such as the CMV immediate-early promoter (described in, for example, U.S. Pat. Nos. 5,168,062 and 5,385,839), promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, and the like.

Alternatively, the invention employs a cellular promoter, i.e., a promoter that drives expression of a cellular protein. Preferred cellular promoters for use in the invention will depend on the desired expression profile to produce the antigen(s). In one aspect, the cellular promoter is preferably a constitutive promoter that works in a variety of cell types, such as immune cells described herein. Suitable constitutive promoters can drive expression of genes encoding transcription factors, housekeeping genes, or structural genes common to eukaryotic cells. For example, the Ying Yang 1 (YY1) transcription factor (also referred to as NMP-1, NF-E1, and UCRBP) is a ubiquitous nuclear transcription factor that is an intrinsic component of the nuclear matrix (Guo et al., PNAS, 92, 10526-10530 (1995)). While these promoters are considered constitutive promoters, it is understood in the art that constitutive promoters can be upregulated. Promoter analysis shows that the elements critical for basal transcription reside from −277 to +475 of the YY1 gene relative to the transcription start site from the promoter, and include a TATA and CCAAT box. JEM-1 (also known as HGMW and BLZF-1) also is a ubiquitous nuclear transcription factor identified in normal and tumor tissues (Tong et al., Leukemia, 12(11), 1733-1740 (1998), and Tong et al., Genomics, 69(3), 380-390 (2000)). JEM-1 is involved in cellular growth control and maturation, and can be upregulated by retinoic acids. Sequences responsible for maximal activity of the JEM-1 promoter have been located at −432 to +101 of the JEM-1 gene relative the transcription start site of the promoter. Unlike the YY1 promoter, the JEM-1 promoter does not comprise a TATA box. The ubiquitin promoter, specifically UbC, is a strong constitutively active promoter functional in several species. The UbC promoter is further characterized in Marinovic et al., J. Biol. Chem., 277(19), 16673-16681 (2002).

The promoter also can be a regulatable promoter, i.e., a promoter that is up- and/or down-regulated in response to appropriate signals. The use of a regulatable promoter or expression control sequence is particularly applicable to DNA vaccine development as antigenic proteins, including viral and parasite antigens, frequently are toxic to cell lines used to produce the gene transfer vector. In one embodiment, the regulatory sequences operably linked to the antigen-encoding nucleic acid sequence include components of the tetracycline expression system, e.g., tet operator sites. For instance, the antigen-encoding nucleic acid sequence is operably linked to a promoter which is operably linked to one or more tet operator sites. A gene transfer vector comprising such an expression cassette can be propagated in a cell line which comprises a nucleic acid sequence encoding a tet repressor protein. By producing the tet repressor protein in the cell line, antigen production is inhibited and propagation proceeds without any associated antigen-mediated toxicity. Suitable regulatable promoter systems also include, but are not limited to, the IL-8 promoter, the metallothionine inducible promoter system, the bacterial lacZYA expression system, and the T7 polymerase system. Further, promoters that are selectively activated at different developmental stages (e.g., globin genes are differentially transcribed from globin-associated promoters in embryos and adults) can be employed. The promoter sequence can contain at least one regulatory sequence responsive to regulation by an exogenous agent. The regulatory sequences are preferably responsive to exogenous agents such as, but not limited to, drugs, hormones, radiation, or other gene products.

The promoter can be a tissue-specific promoter, i.e., a promoter that is preferentially activated in a given tissue and results in expression of a gene product in the tissue where activated. A tissue-specific promoter suitable for use in the invention can be chosen by the ordinarily skilled artisan based upon the target tissue or cell-type. Preferred tissue-specific promoters for use in the inventive method are specific to immune cells, such as the dendritic-cell specific Dectin-2 promoter described in Morita et al., Gene Ther., 8, 1729-37 (2001).

In yet another embodiment, the promoter can be a chimeric promoter. A promoter is “chimeric” in that it comprises at least two nucleic acid sequence portions obtained from, derived from, or based upon at least two different sources (e.g., two different regions of an organism's genome, two different organisms, or an organism combined with a synthetic sequence). Preferably, the two different nucleic acid sequence portions exhibit less than about 40%, more preferably less than about 25%, and even more preferably less than about 10% nucleic acid sequence identity to one another (which can be determined by methods described elsewhere herein). Any suitable chimeric promoter can be used in the inventive method. Preferably, the chimeric promoter is comprised of a functional portion of a viral promoter and a functional portion of a cellular promoter. More preferably, the chimeric promoter comprises a functional portion of a viral promoter and a functional portion of a cellular promoter that is radiation-inducible. Most preferably, the chimeric promoter comprises a functional portion of a CMV promoter and a functional portion of an EGR-1 promoter (i.e., a chimeric “CMV/EGR-1” promoter). The functional portion of the CMV promoter preferably is derived from a human CMV, and more particularly from the human CMV immediate early (IE) promoter/enhancer region (see, e.g., U.S. Pat. Nos. 5,168,062 and 5,385,839). In addition, the functional portion of the EGR-1 promoter preferably comprises one or more CArG domains of an EGR-1 promoter, as described in, for example, U.S. Pat. Nos. 6,579,522 and 6,605,712. In a particularly preferred embodiment of the invention, the chimeric promoter comprises a functional portion of the CMV IE enhancer/promoter region, and an EGR-1 promoter comprising six CArG domains. In this manner, the portion of the CMV IE enhancer/promoter region functions as an enhancer for the EGR-1 promoter. Chimeric promoters can be generated using standard molecular biology techniques, such as those described in Sambrook et al., supra, and Ausubel et al., supra.

A “functional portion” of a promoter is any portion of a promoter that measurably promotes, enhances, or controls expression (typically transcription) of an operatively linked nucleic acid. Such regulation of expression can be measured via RNA or protein detection by any suitable technique, and several such techniques are known in the art. Examples of such techniques include Northern analysis (see, e.g., Sambrook et al., supra, and McMaster and Carmichael, PNAS, 74, 4835-4838 (1977)), RT-PCR (see, e.g., U.S. Pat. No. 5,601,820, and Zaheer et al., Neurochem Res., 20, 1457-1463 (1995)), in situ hybridization methods (see, e.g., U.S. Pat. Nos. 5,750,340 and 5,506,098), antibody-mediated techniques (see, e.g., U.S. Pat. Nos. 4,376,110, 4,452,901, and 6,054,467), and promoter assays utilizing reporter gene systems such as the luciferase gene (see, e.g., Taira et al., Gene, 263, 285-292 (2001)). Eukaryotic expression systems in general are further described in Sambrook et al., supra.

A promoter can be selected for use in the method of the invention by matching its particular pattern of activity with the desired pattern and level of expression of the antigen(s). For example, the gene transfer vector can comprise two or more nucleic acid sequences that encode different antigens and are operably linked to different promoters displaying distinct expression profiles. For example, a first promoter is selected to mediate an initial peak of antigen production, thereby priming the immune system against an encoded antigen. A second promoter is selected to drive production of the same or different antigen such that expression peaks several days after that of the first promoter, thereby “boosting” the immune system against the antigen. Alternatively, a chimeric promoter can be constructed which combines the desirable aspects of multiple promoters. For example, a CMV-RSV hybrid promoter combining the CMV promoter's initial rush of activity with the RSV promoter's high maintenance level of activity is especially preferred for use in many embodiments of the inventive method. In that antigens can be toxic to eukaryotic cells, it may be advantageous to modify the promoter to decrease activity in cell lines used to propagate the gene transfer vector.

To optimize protein production, preferably the nucleic acid sequence encoding the antigen further comprises a polyadenylation site following the coding sequence of the antigen-encoding nucleic acid sequence. Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), and the papillomaviruses, including human papillomaviruses and BPV (Bovine Papilloma Virus). A preferred polyadenylation sequence is the SV40 (Human Sarcoma Virus-40) polyadenylation sequence. Also, preferably all the proper transcription signals (and translation signals, where appropriate) are correctly arranged such that the nucleic acid sequence is properly expressed in the cells into which it is introduced. If desired, the nucleic acid sequence also can incorporate splice sites (i.e., splice acceptor and splice donor sites) to facilitate mRNA production.

If the antigen-encoding nucleic acid sequence encodes a processed or secreted protein or peptide, or a protein that acts intracellularly, preferably the antigen-encoding nucleic acid sequence further comprises the appropriate sequences for processing, secretion, intracellular localization, and the like. The antigen-encoding nucleic acid sequence can be operably linked to a signal sequence, which targets a protein to cellular machinery for secretion. Appropriate signal sequences include, but are not limited to, leader sequences for immunoglobulin heavy chains and cytokines, (see, e.g., Ladunga, Current Opinions in Biotechnology, 11, 13-18 (2000)). Other protein modifications can be required to secrete a protein from a host cell, which can be determined using routine laboratory techniques. Preparing expression constructs encoding antigens and signal sequences is further described in, for example, U.S. Pat. No. 6,500,641. Methods of secreting non-secretable proteins are further described in, for example, U.S. Pat. No. 6,472,176, and International Patent Application Publication WO 02/48377.

The antigen encoded by the nucleic acid sequence of the gene transfer vector also can be modified to attach or incorporate the antigen on the host cell surface. In this respect, the antigen can comprise a membrane anchor, such as a gpi-anchor, for conjugation onto the cell surface. A transmembrane domain can be fused to the antigen to incorporate a terminus of the antigen protein into the cell membrane. Other strategies for displaying peptides on a cell surface are known in the art and are appropriate for use in the context of the invention.

It is believed that a portion of the shaft region of a subgroup C adenovirus fiber protein provides a mechanism by which adenovirus binds to and enters specific cell types, such as, for example, dendritic cells and tumor cells. Thus, the inventive gene transfer vector further comprises an amino acid sequence comprising at least three (e.g., three or more, five or more, ten or more, twenty or more, thirty or more, forty or more, or even fifty or more) contiguous amino acids of a shaft region of a subgroup C adenovirus fiber protein. The fiber protein of adenovirus is a trimer (Devaux et al., J. Molec. Biol., 215, 567-588 (1990)) consisting of a tail, a shaft, and a knob. The fiber shaft region is composed of repeating 15 amino acid motifs, which are believed to form two alternating β-strands and β-bends (Green et al., EMBO J., 2, 1357-1365 (1983), and Chroboczek et al., In P. B. W. Doerfler, ed., The Molecular Repertoire of Adenoviruses, Vol. 1, Springer-Verlag, Berlin, Germany, pp.163-200 (1995)). The overall length of the fiber shaft region and the number of 15 amino-acid repeats differ between adenoviral serotypes. For example, the Ad2 fiber shaft is 37 nanometers long and contains 22 repeats, whereas the Ad3 fiber is 11 nanometers long and contains 6 repeats. A receptor binding domain of the fiber protein is localized in the knob region encoded by the last 200 amino acids of the protein (Henry et al., J. Virology, 68(8), 5239-5246 (1994)). The regions necessary for trimerization are also located in the knob region of the protein (Henry et al. (1994), supra). A deletion mutant lacking the last 40 amino acids of the knob region of the fiber protein does not trimerize and also does not bind to penton base (Novelli et al., Virology, 185, 365-376 (1991)). Thus, trimerization of the fiber protein is necessary for penton base binding to the fiber. Nuclear localization signals that direct the protein to the nucleus to form viral particles following its synthesis in the cytoplasm are located in the N-terminal region of the protein (Novelli et al. (1991), supra). The fiber, together with the hexon, determine the serotype specificity of the adenovirus (Watson et al., J. Gen. Virol., 69, 525-535 (1988)).

The amino acid sequence comprising at least three contiguous amino acids of a shaft region preferably is derived from a subgroup C adenovirus fiber protein. In this regard, the amino acid sequence can be derived from any suitable subgroup C adenovirus serotype. Suitable subgroup C adenovirus serotypes include serotypes 1, 2, 5, and 6. The amino acid sequence preferably comprises at least three (e.g., 3, 10, 20, 30, 40, 50, or more) contiguous amino acids of a shaft region of a serotype 2 or serotype 5 adenovirus fiber protein. The amino acid sequence can be the entire shaft region of a subgroup C (especially a serotype 2 or serotype 5) adenovirus fiber protein. The amino acid sequence preferably comprises from about 3 to about 50 contiguous amino acids (e.g., 15, 20, 30, 40, or 50 amino acids) of a shaft region of a subgroup C (especially a serotype 2 or serotype 5) adenovirus fiber protein. The amino acid sequence more preferably comprises from about 3 to about 20 contiguous amino acids (e.g., about 5, 10, 15, or 20 amino acids) of a shaft region of a subgroup C (especially a serotype 2 or serotype 5) adenovirus fiber protein. Most preferably, the amino acid sequence comprises the amino acid residues lysine-lysine-threonine-lysine (KKTK) (SEQ ID NO: 1). For example, the amino acid sequence can consist of the sequence KKTK. Alternatively, SEQ ID NO: 1 can be part of a larger amino acid sequence that is included in the inventive gene transfer vector. In another embodiment, the amino acid sequence can comprise a fragment of SEQ ID NO: 1, such as, for example, KKT (SEQ ID NO: 2) or KTK (SEQ ID NO:3). The KKTK motif in the shaft region of Ad5 is known to bind glycosaminoglycans (GAGs) (e.g., heparin and heparan sulfate proteoglycans) (see, e.g., Hileman et al., Bioessays, 20, 156-67 (1998), and Smith et al., Mol Ther., 5, 770-9 (2002)). GAGs are long unbranched polysaccharides containing a repeating disaccharide unit. The disaccharide units contain either of two modified sugars. The majority of GAGs in a human are linked to core proteins, forming proteoglycans (also called mucopolysaccharides). GAGs are located primarily on the surface of cells or in the extracellular matrix (ECM). The amino acid sequences of the fiber protein of a serotype 2 adenovirus and a serotype 5 adenovirus are disclosed in Chroboczek et al., Virology, 161, 549-54 (1987).

One of ordinary skill in the art will appreciate that the immune response elicited by the gene transfer vector once administered to the mammal (e.g., a human) will depend upon the location of the amino acid sequence comprising at least three contiguous amino acids of a shaft region of a subgroup C adenovirus fiber protein within the gene transfer vector. In this respect, the amino acid sequence can be present in any suitable location within the gene transfer vector. In this regard, the amino acid sequence can be located on the surface of the gene transfer vector to maximize recognition by the host immune system. When the gene transfer vector is a viral vector, for example, the amino acid sequence preferably is located on or within the virus capsid, as a result of modification of the capsid proteins. Such modifications include, for example, generating chimeric capsid proteins which comprise the amino acid sequence. Such chimeric capsid proteins can be generated using routine molecular biology and recombinant DNA techniques (see, e.g., Sambrook et al., supra). Alternatively, it is also possible to covalently attach the amino acid sequence to a capsid protein of a viral gene transfer vector by chemical modification, such as by chemical cross-linkage. Chemical cross-linkage can occur using available sulfhydral or amide groups on the viral vector. Such groups also can be found or introduced into the the amino acid sequence comprising at least three contiguous amino acids of a shaft region of a subgroup C adenovirus fiber protein. Examples of suitable cross-linking agents include, for example, 4-azidobenzoic acid (3-sulfo-N-succinimidyl) ester Sodium salt (Sulfo-HSAB), 1,4-bis[3-(2-pyridyldithio)propionamido]butane, and bis[2-(N-succinimidyl-oxycarbonyloxy)ethyl]sulfone. Other methods for chemically cross-linking proteins are known in the art.

The invention further provides a method of inducing an immune response in a mammal comprising administering to the mammal a conjugate comprising (a) at least one antigen which induces an immune response in the mammal, and (b) an amino acid sequence comprising at least three contiguous amino acids of a shaft region of a subgroup C adenovirus fiber protein, wherein the amino acid sequence is not an intact adenovirus. Descriptions of the antigen and the amino acid sequence comprising at least three contiguous amino acids of a subgroup C adenoviral fiber protein set forth above in connection with other embodiments of the invention also are applicable to those same aspects of the aforesaid inventive method of inducing an immune response.

A “conjugate” is a molecule that is generated via coupling of two or more separate molecules. The inventive conjugate preferably comprises the antigen chemically coupled to the amino acid sequence of the shaft region of a subgroup C adenovirus. The antigen can be chemically coupled to the amino acid sequence by any suitable chemical bond, and preferably is coupled to the amino acid sequence via covalent bonds. Suitable chemical bonds are well known in the art and include disulfide bonds, acid labile bonds, photolabile bonds, peptidase labile bonds, thioether bonds, and esterase labile bonds. Such conjugates can be produced using routine molecular biology techniques, such as those described in Sambrook et al., supra.

Alternatively, the conjugate can be a fusion protein comprising the antigen and the amino acid sequence of the shaft region of a subgroup C adenovirus. The fusion protein can be generated using routine molecular biology techniques, such as restriction enzyme or recombinational cloning techniques (see, e.g., Gateway™ cloning system (Invitrogen) and U.S. Pat. Nos. 5,314,995 and 5,994,104). When the conjugate is a fusion protein, the invention further provides a nucleic acid molecule encoding the fusion protein. In this respect, “nucleic acid molecule” is intended to encompass a polymer of DNA or RNA, i.e., a polynucleotide, which can be single-stranded or double-stranded and which can contain non-natural or altered nucleotides. In one embodiment, the nucleic acid molecule can lack introns or portions thereof. The nucleic acid molecule preferably is DNA. The nucleic acid molecule may be isolated or purified from any suitable source. The nucleic acid molecule also may be chemically synthesized by methods known in the art.

When the conjugate is a fusion protein, the fusion protein also can include additional peptide sequences which act to promote stability, purification, and/or detection of the fusion protein. For example, a reporter peptide portion (e.g., green fluorescent protein (GFP), β-galactosidase, or a detectable domain thereof) can be incorporated in the fusion protein. Purification-facilitating peptide sequences include those derived or obtained from maltose binding protein (MBP), glutathione-S-transferase (GST), or thioredoxin (TRX). The fusion protein also or alternatively can be tagged with an epitope which can be antibody purified (e.g., the Flag epitope, which is commercially available from Kodak (New Haven, Conn.)), a hexa-histidine peptide, such as the tag provided in a pQE vector available from QIAGEN, Inc. (Chatsworth, Calif.), or an HA tag (as described in, e.g., Wilson et al., Cell, 37, 767 (1984)).

In the methods of the invention, the gene transfer vector or conjugate preferably is administered to a mammal (e.g., a human), wherein the antigen induces an immune response against the antigen. The immune response can be a humoral immune response, a cell-mediated immune response, or, desirably, a combination of humoral and cell-mediated immunity. Ideally, the immune response provides protection upon subsequent challenge with the antigen. However, protective immunity is not required in the context of the invention. The inventive method further can be used for antibody production and harvesting.

To enhance the immune response generated against the antigen, the gene transfer vector can further comprise a nucleic acid sequence that encodes an immune stimulator, such as a cytokine, a chemokine, or a chaperone. Cytokines include, for example, Macrophage Colony Stimulating Factor (e.g., GM-CSF), Interferon Alpha (IFN-α), Interferon Beta (IFN-β), Interferon Gamma (IFN-γ), interleukins (IL-1, IL-2, IL-4, IL-5, IL-6, IL-8, IL-10, IL-12, IL-13, IL-15, IL-16, and IL-18), the TNF family of proteins, Intercellular Adhesion Molecule-1 (ICAM-1), Lymphocyte Function-Associated antigen-3 (LFA-3), B7-1, B7-2, FMS-related tyrosine kinase 3 ligand, (Flt3L), vasoactive intestinal peptide (VIP), and CD40 ligand. Chemokines include, for example, B Cell-Attracting chemokine-1 (BCA-1), Fractalkine, Melanoma Growth Stimulatory Activity protein (MGSA), Hemofiltrate CC chemokine 1 (HCC-1), Interleukin 8 (IL8), Interferon-stimulated T-cell alpha chemoattractant (1-TAC), Lymphotactin, Monocyte Chemotactic Protein 1 (MCP-1), Monocyte Chemotactic Protein 3 (MCP-3), Monocyte Chemotactic Protein 4 (MCP-4), Macrophage-Derived Chemokine (MDC), a macrophage inflammatory protein (MIP), Platelet Factor 4 (PF4), Regulated upon Activation, Normal T-cell Expressed, and presumably Secreted (RANTES), breast and kidney cell chemokine (BRAK), eotaxin, exodus 1-3, and the like. Chaperones include, for example, the heat shock proteins Hsp170, Hsc70, and Hsp40. Cytokines and chemokines are generally described in the art, including the Invivogen catalog (2002), San Diego, Calif. Likewise, the inventive conjugate can comprise any one or more of the aforementioned immune stimulators.

Multiple gene transfer vectors can be administered to the mammal, each gene transfer vector comprising one or more nucleic acid sequences encoding one or more antigens and/or immunomodulators. Similarly, multiple conjugates can be administered to the mammal, each conjugate comprising one or more antigens and/or immunomodulators. If the gene transfer vector comprises more than one antigen-encoding nucleic acid sequence, two or more nucleic acid sequences can be operably linked to the same promoter (e.g., to form a bicistronic sequence), two or more nucleic acid sequences can be operably linked to separate promoters of the same type (e.g., the CMV promoter), or two or more nucleic acid sequences can be operably linked to separate and different promoters (e.g., the CMV promoter and β-actin promoter). The multiple gene transfer vectors can include two or more gene transfer vector constructs encoding different antigens, different epitopes of the same antigenic protein, the same antigenic protein derived from different species or clades of microorganism, antigens from different microorganisms, and the like. Similarly, the multiple conjugates also can include two or more conjugates comprising different antigens, different epitopes of the same antigenic protein, the same antigenic protein derived from different species or clades of microorganism, antigens from different microorganisms, and the like. In will be appreciated that, in some embodiments, administering a “cocktail” of gene transfer vectors and/or conjugates having different antigens or different epitopes of the same antigen can provide a more effective immune response than administering a single gene transfer vector clone or a single conjugate to a mammal.

Likewise, administering the gene transfer vector or the conjugate can be one component of a multistep regimen for inducing an immune response in a mammal. In particular, the inventive method can represent one arm of a prime and boost immunization regimen. The inventive method, for example, can comprise administering to the mammal a priming gene transfer vector comprising a nucleic acid sequence encoding at least one antigen prior to administering the inventive gene transfer vector. The antigen of the priming gene transfer vector or conjugate can be the same or different from the antigen of the subsequently administered gene transfer vector or conjugate. The subsequently administered gene transfer vector or conjugate is then administered to boost the immune response to a given pathogen. More than one boosting composition comprising the gene transfer vector or conjugate can be provided in any suitable timeframe (e.g., at least about 1 week, 2 weeks, 4 weeks, 8 weeks, 12 weeks, 16 weeks, or more following priming) to maintain immunity.

The priming gene transfer vector or conjugate need not be a gene transfer vector or conjugate as otherwise described herein for use in the inventive methods. For example, any gene transfer vector can be employed as a priming gene transfer vector, including, but not limited to, a plasmid, a retrovirus, an adeno-associated virus, a vaccinia virus, a herpesvirus, or a bacteriophage. Ideally, the priming gene transfer vector is a plasmid. Alternatively, an immune response can be primed or boosted by administration of the antigen itself, e.g., an antigenic protein, inactivated pathogen, and the like.

Any route of administration can be used to deliver the gene transfer vector or the conjugate to the mammal. Indeed, although more than one route can be used to administer the gene transfer vector or the conjugate, a particular route can provide a more immediate and more effective reaction than another route. Preferably, the gene transfer vector or the conjugate is administered via intramuscular injection. A dose of gene transfer vector or conjugate also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The gene transfer vector or conjugate can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the gene transfer vector or conjugate. The gene transfer vector or conjugate also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of gene transfer vector or conjugate administered to the mammal will depend on a number of factors, including the type of gene transfer vector (e.g. virus or liposome) or conjugate (e.g., fusion protein), the size of a target tissue, the extent of any side-effects, the particular route of administration, and the like. The dose ideally comprises an “effective amount” of gene transfer vector or conjugate, i.e., a dose of gene transfer vector or conjugate which provokes a desired immune response in the mammal. The desired immune response can entail production of antibodies, protection upon subsequent challenge, immune tolerance, immune cell activation, and the like.

The gene transfer vector or conjugate desirably is administered in a composition, preferably a physiologically acceptable (e.g., pharmaceutically acceptable) composition, which comprises a carrier, preferably a physiologically (e.g., pharmaceutically) acceptable carrier and the gene transfer vector(s) or conjugate(s). Any suitable carrier can be used within the context of the invention, and such carriers are well known in the art. The choice of carrier will be determined, in part, by the particular site to which the composition is to be administered and the particular method used to administer the composition. The composition can optionally be sterile or sterile with the exception of the inventive gene transfer vector or conjugate.

Suitable carriers and their formulations are described in A. R. Gennaro, ed., Remington: The Science and Practice of Pharmacy (19th ed.), Mack Publishing Company, Easton, Pa. (1995). Pharmaceutical carriers include sterile water, saline, Ringer's solution, dextrose solution, and buffered solutions at physiological pH. Typically, an appropriate amount of a pharmaceutically acceptable salt is used in the formulation to render the formulation isotonic. The pH of the formulation is preferably from about 5 to about 8 (e.g., about 5.5, about 6, about 6.5, about 7, about 7.5, and ranges thereof). More preferably, the pH is about 7 to about 7.5. Further carriers include sustained-release preparations, such as semipermeable matrices of solid hydrophobic polymers containing the gene transfer vector or the conjugate, which matrices are in the form of shaped articles (e.g., films, liposomes, or microparticles). It will be apparent to those persons skilled in the art that certain carriers may be more preferable depending upon, for instance, the route of administration and concentration of composition being administered.

Compositions (e.g., pharmaceutical compositions) comprising the gene transfer vector or the conjugate can include carriers, thickeners, diluents, buffers, preservatives, surface active agents, and the like. The compositions can also include one or more active ingredients, such as antimicrobial agents, anti-inflammatory agents, anesthetics, and the like.

The composition comprising the gene transfer vector or conjugate can be administered in any suitable manner depending on whether local or systemic treatment is desired, and on the area to be treated. Administration can be topical (including ophthalmical, vaginal, rectal, transdermal, and the like), oral, by inhalation, or parenteral (including by intravenous drip or subcutaneous, intracavity, intraperitoneal, or intramuscular injection). Inhalation administration refers to the delivery of the compositions into the nose and nasal passages through one or both of the nares and can comprise delivery by a spraying mechanism or droplet mechanism, or through aerosolization of the nucleic acid, vector, or fusion protein. Delivery can also be directly to any area of the respiratory system (e.g., lungs) via intubation.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve the preparation of a slow-release or sustained-release system, such that a constant dosage is maintained. Preparations for parenteral administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives also can be present such as, for example, antimicrobials, anti-oxidants, chelating agents, inert gases and the like.

Formulations for topical administration may include ointments, lotions, creams, gels, drops, suppositories, sprays, liquids, and powders. Conventional pharmaceutical carriers, aqueous, powder, or oily bases, thickeners, and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable.

Some of the compositions can potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines.

The gene transfer vector or conjugate can be administered with a pharmaceutically acceptable carrier and can be delivered to the mammal's cells in vivo and/or ex vivo by a variety of mechanisms well-known in the art (e.g., uptake of naked DNA, liposome fusion, intramuscular injection of DNA via a gene gun, endocytosis, and the like).

If ex vivo methods are employed, cells or tissues can be removed and maintained outside the body according to standard protocols known in the art. The compositions can be introduced into the cells via any gene transfer mechanism, such as calcium phosphate mediated gene delivery, electroporation, microinjection, or proteoliposomes. The transduced cells then can be infused (e.g., with a pharmaceutically acceptable carrier) or homotopically transplanted back into the mammal per standard methods for the cell or tissue type. Standard methods are known for transplantation or infusion of various cells into a mammal.

In addition, one of ordinary skill in the art will appreciate that the gene transfer vector or the conjugate can be present in a composition with other therapeutic or biologically-active agents. For example, factors that control inflammation, such as ibuprofen or steroids, can be part of the composition to reduce swelling and inflammation associated with in vivo administration of the gene transfer vector or the conjugate. As discussed herein, immune system stimulators can be administered to enhance any immune response to the antigen. Antibiotics, i.e., microbicides and fungicides, can be present to treat existing infection and/or reduce the risk of future infection, such as infection associated with gene transfer procedures.

The invention further provides a gene transfer vector comprising (a) a nucleic acid sequence encoding a protein, and (b) an amino acid sequence comprising at least three contiguous amino acids of a shaft region of a subgroup C adenovirus fiber protein, wherein the gene transfer vector is not an adenoviral vector. Descriptions of the gene transfer vector and the amino acid sequence comprising at least three contiguous amino acids of a subgroup C adenoviral fiber protein set forth above in connection with other embodiments of the invention also are applicable to those same aspects of the aforesaid inventive gene transfer vector. The gene transfer vector can by any suitable gene transfer vector set forth above (e.g., a viral vector, a liposome, or a virus-like particle). The gene transfer vector preferably is a viral vector, but is not an adenoviral vector.

The nucleic acid sequence encoding the protein can be obtained from any source, e.g., isolated from nature, synthetically generated, isolated from a genetically engineered organism, and the like. An ordinarily skilled artisan will appreciate that any type of nucleic acid sequence (e.g., DNA, RNA, and cDNA) that can be inserted into a gene transfer vector can be used in connection with the invention.

The nucleic acid sequence of the inventive gene transfer vector preferably encodes a protein that is toxic to mammalian (e.g., human) cells (i.e., a “cytotoxic” protein). Suitable toxic proteins include, bacterial toxins, viral toxins, fungal toxins, or toxins produced by a parasitic agent, plant toxins, cytotoxic drugs (e.g., chemotherapeutic drugs), and radionuclides. Suitable bacterial toxins include, for example, Aeromonas hydrophila aerolysin toxin, Escherichia coli hemolysin toxin, the enterotoxins, exfoliative toxins, toxic-shock toxins, and α-toxin of Staphyloccocus aureus, Streptococcus pyogenes streptolysin O toxin and pyrogenic exotoxins, diphtheria toxin, Bacillus anthracis edema factor and lethal factor, Bordetella pertussis dermonecrotic toxin and pertussis toxin, cholera toxin, tetanus toxin, and Psuedomonas aeruginosa exotoxin A. Suitable viral toxins include, for example, herpes simplex virus thymidine kinase (TK), and any toxin produced by a virus from the family Hepadnaviridae, Parvoviridae (e.g., adeno-associated viruses (AAVs)), Papovaviridae (e.g., the papillomaviruses), Adenoviridae (e.g., human adenovirus), Picornaviridae (e.g., hepatitis A virus), Herpesviridae (e.g., the herpes simplex-like viruses), Retroviridae (e.g., HIV), or any other virus family described herein. The protein encoded by the nucleic acid sequence of the inventive gene transfer vector, however, is not limited to these exemplary proteins. Indeed, any protein that is cytotoxic to a mammalian cell, preferably a human cell is within the scope of the invention.

In another embodiment, the nucleic acid sequence of the inventive gene transfer vector can encode an antigen. In this respect, the nucleic acid sequence can encode any suitable antigen, such as those described herein.

The nucleic acid sequence of the inventive gene transfer vector preferably encodes a secreted protein, e.g., a protein that is naturally secreted by the infected cell. By “secreted protein” is meant any peptide, polypeptide, or portion thereof, which is released by a cell into the extracellular environment. The nucleic acid sequence also can encode a protein that is not naturally secreted by the cell, but which is released by cell lysis induced by gene transfer vector (e.g., viral vector) transduction. Alternatively, the nucleic acid sequence can encode a protein that is not naturally secreted by the cell (i.e., a non-secretable protein), but which comprises a signal peptide that facilitates protein secretion (see, e.g., U.S. Pat. No. 6,472,176). In this manner, for example, the nucleic acid sequence encodes an endoplasmic reticulum (ER) localization signal peptide and the non-secretable protein. The ER localization signal peptide functions to direct DNA, RNA, and/or a protein to the membrane of the endoplasmic reticulum, wherein a protein is expressed and targeted for secretion. The ER localization signal peptide desirably functions to increase the secretion (i.e., the secretion potential) by a cell of (i) proteins that are not normally secreted (i.e., secretable) by the cell and/or (ii) proteins that are normally secreted by a cell, but in low (i.e., less than desired) quantities. The ER localization signal peptide encoded by the polynucleotide can be any suitable ER localization signal peptide or polypeptide (i.e., protein). For example, the ER localization signal peptide encoded by the nucleic acid sequence can be a peptide or polypeptide (i.e., protein) selected from the group consisting of nerve growth factor (NGF), immunoglobulin (Ig) (e.g., an Ig κ chain leader sequence), and midkine (MK), or a portion thereof. Suitable ER localization signal peptides also include those described in Ladunga, Current Opinions in Biotechnology, 11, 13-18 (2000).

The nucleic acid sequence can encode a tumor necrosis factor (TNF), a vascular endothelial growth factor (VEGF), or a pigment epithelium-derived factor (PEDF). Preferably, the gene transfer vector comprises a nucleic acid sequence coding for a TNF. Nucleic acid sequences encoding a TNF include nucleic acid sequences encoding any member of the TNF family of proteins (e.g., CD40 ligand and Fas ligand). The gene transfer vector preferably comprises a nucleic acid sequence encoding human TNF-α. A nucleic acid sequence coding for TNF is described in U.S. Pat. No. 4,879,226. Alternatively, the nucleic acid sequence can encode a VEGF. The nucleic acid sequence can encode any suitable VEGF isoform, including, but not limited to, VEGF₁₂₁, VEGF₁₄₅, VEGF₁₆₅, VEGF₁₈₉, or VEGF₂₀₆, which are variously described in U.S. Pat. Nos. 5,332,671, 5,240,848, and 5,219,739. Most preferably, because of their higher biological activity, the nucleic acid sequence encodes VEGF₁₂₁ or VEGF₁₆₅, particularly VEGF₁₂₁. A notable difference between VEGF₁₂₁ and VEGF₁₆₅ is that VEGF₁₂₁ does not bind to heparin with a high degree of affinity, as does VEGF₁₆₅. Other suitable VEGF peptides are VEGF-II, VEGF-C, and the like. The nucleic acid sequence also can encode a PEDF. PEDF, also known as early population doubling factor-1 (EPC-1), is a secreted protein having homology to a family of serine protease inhibitors named serpins. PEDF is made predominantly by retinal pigment epithelial cells and is detectable in most tissues and cell types of the body. PEDF has both neurotrophic and anti-angiogenic properties and, therefore, is useful in the treatment and study of a broad array of diseases. Nucleic acid sequences encoding anti-angiogenic derivatives of PEDF, known as SLED proteins (see, e.g., International Patent Application Publication No. WO 99/04806), also can be used in connection with the invention. PEDF is further characterized in International Patent Application Publication Nos. WO 93/24529 and WO 99/04806, and the nucleic acid sequence encoding PEDF is described in U.S. Pat. No. 5,840,686.

The inventive gene transfer vector can comprise one or more additional nucleic acid sequences, each encoding one or more gene products, such that one or more additional proteins are expressed in a host cell. The expression of the additional gene product(s) can be separately regulated by individual expression control sequences, or coordinately regulated by one common expression control sequence. Alternatively, the expression of the additional nucleic acid(s) can be regulated by the same expression control sequence that regulates expression of the previously described nucleic acid sequence encoding the protein; however, any transcription terminating regions present in the nucleic acid encoding the protein would be eliminated to allow for transcriptional read-through of the additional nucleic acid sequence(s). The additional nucleic acid sequence(s) can comprise any suitable expression control sequence(s) and any suitable transcription-termination region(s) discussed herein in connection with the previously described nucleic acid sequence encoding the protein.

The nucleic acid sequence can encode any variant, homolog, or functional portion of the aforementioned proteins. A variant of the protein can include one or more mutations (e.g., point mutations, deletions, insertions, etc.) from a corresponding naturally occurring protein. By “naturally occurring” is meant that the protein can be found in nature and has not been synthetically modified. When mutations are introduced in the nucleic acid sequence encoding the protein, such mutations desirably will effect a substitution in the encoded protein whereby codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and/or codons encoding neutral non-polar residues (A, F, I, L, M, P, V, and W) are substituted with codons encoding neutral non-polar residues. In addition, a homolog of the protein can be any peptide, polypeptide, or portion thereof, that is more than about 70% identical (preferably more than about 80% identical, more preferably more than about 90% identical, and most preferably more than about 95% identical) to the protein at the amino acid level. The degree of amino acid identity can be determined using any method known in the art, such as the BLAST sequence database. Furthermore, a homolog of the protein can be any peptide, polypeptide, or portion thereof, which hybridizes to the protein under at least moderate, preferably high, stringency conditions. Exemplary moderate stringency conditions include overnight incubation at 370° C. in a solution comprising 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., supra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at (i) 42° C. in 0.2×SSC, (ii) at 55° C. in 50% formamide and (iii) at 55° C. in 0.1×SSC (preferably in combination with EDTA). Additional details and an explanation of stringency of hybridization reactions are provided in, e.g., Ausubel et al., supra. A “functional portion” is any portion of a protein that retains the biological activity of the naturally occurring, full-length protein at measurable levels.

The expression of the nucleic acid sequence is controlled by any suitable promoter. Ideally, the nucleic acid sequence is operably linked to a promoter and a polyadenylation sequence as described herein.

The invention also provides a conjugate comprising (a) a protein or a non-proteinaceous molecule, and (b) an amino acid sequence comprising at least three contiguous amino acids of a shaft region of a subgroup C adenovirus fiber protein, wherein when the conjugate comprises a protein, the conjugate is not an adenovirus. Descriptions of the conjugate and the amino acid sequence comprising at least three contiguous amino acids of a shaft region of a subgroup C adenovirus fiber protein set forth above in connection with other embodiments of the invention also are applicable to those same aspects of the aforesaid inventive conjugate. In this respect, the conjugate preferably comprises the protein or non-proteinaceous molecule chemically coupled to the amino acid sequence of the shaft region of a subgroup C adenovirus. The protein or non-proteinaceous molecule can be chemically coupled to the amino acid sequence by any suitable chemical bond, and preferably is coupled to the amino acid sequence via covalent bonds, such as those described herein. More preferably, the inventive conjugate is a fusion protein.

When the conjugate comprises a protein, the conjugate preferably is not an adenovirus, especially an encapsidated adenovirus. An encapsidated adenovirus comprises an adenoviral genome contained within an adenovirus capsid. An encapsidated adenovirus can be replication-competent, conditionally replication-deficient, or replication-deficient. A conditionally replication-deficient adenovirus is engineered to replicate under conditions pre-determined by the practitioner. A replication-deficient adenovirus comprises an adenoviral genome that lacks at least one replication-essential gene function (i.e., such that the adenoviral vector does not replicate in typical host cells, especially those in a human patient that could be infected by the adenoviral vector).

In one embodiment, the conjugate comprises one or more proteins. The protein can be any suitable protein described herein. Suitable proteins include, for example, an antigen, a cytotoxic protein (e.g., a bacterial or viral toxin), a secreted protein (e.g., a TNF, VEGF, or PEDF), and a protein that enhances immune responses (e.g., TNF, GMCSF or IL-12).

In another embodiment, the conjugate comprises a non-proteinaceous molecule. The conjugate can comprise any suitable non-proteinaceous molecule. Suitable non-proteinaceous molecules include, for example, carbohydrates, lipids, and non-proteinaceous small molecules. By “small molecule” is meant any molecule that is not considered a macromolecule, which includes a nucleic acid, a carbohydrate, or a lipid. Small molecules are a diverse group of natural and synthetic substances that generally have a low molecular weight. For example, a small molecule typically has a molecular weight of less than about 1,000 (e.g., less than about 700, 500, or 300). Small molecules can be isolated from natural sources such as plants, fungi, or microbes, or they can be synthesized by organic chemistry. Most conventional pharmaceuticals, such as aspirin, penicillin, and chemotherapeutics, are small molecules. The non-proteinaceous molecule preferably is a hapten. A hapten is a low molecular weight molecule which contains an antigenic determinant, but which itself is not antigenic unless complexed with an antigenic carrier. Examples of suitable haptens for use in the invention include dinitrophenols, phosphrylcholine, and dextran.

The invention provides a method of delivering a protein or a non-proteinaceous molecule to a cell. The method comprises contacting the cell with a gene transfer or conjugate as described herein.

The protein or non-proteinaceous molecule can be delivered to any suitable cell. As previously mentioned, it is believed that a portion of the shaft region of a subgroup C adenovirus fiber protein is responsible for targeting adenovirus to specific cell types (e.g., immune cells and tumor cells). In accordance with the inventive method, the protein or non-proteinaceous molecule can be delivered to immune cells, preferably antigen presenting cells, such as dendritic cells, monocytes, and macrophages.

When dendritic cells are the desired target cell, the gene transfer vector or the conjugate, by way of the amino acid sequence of a subgroup C adenovirus fiber shaft region, recognizes a protein typically found on dendritic cell surfaces such as adhesion proteins, chemokine receptors, complement receptors, co-stimulation proteins, cytokine receptors, high level antigen presenting molecules, homing proteins, marker proteins, receptors for antigen uptake, signaling proteins, virus receptors, etc. Examples of such potential ligand-binding sites in dendritic cells include αᵥβ₃ integrins, αᵥβ₅ integrins, 2A1, 7-TM receptors, CD1, CD11a, CD11b, CD11c, CD21, CD24, CD32, CD4, CD40, CD44 variants, CD46, CD49d, CD50, CD54, CD58, CD64, ASGPR, CD80, CD83, CD86, E-cadherin, integrins, M342, MHC-I, MHC-II, MIDC-8, MMR, OX62, p200-MR6, p55, S100, TNF-R, etc. When dendritic cells are targeted, the amino acid sequence comprising at least three contiguous amino acids of a shaft region of a subgroup C adenovirus fiber protein preferably recognizes the CD40 cell surface protein.

When macrophages are the desired target, the gene transfer vector or the conjugate, by way of the amino acid sequence of a subgroup C adenovirus fiber shaft region, recognizes a protein typically found on macrophage cell surfaces, such as phosphatidylserine receptors, vitronectin receptors, integrins, adhesion receptors, receptors involved in signal transduction and/or inflammation, markers, receptors for induction of cytokines, or receptors up-regulated upon challenge by pathogens, members of the group B scavenger receptor cysteine-rich (SRCR) superfamily, sialic acid binding receptors, members of the Fc receptor family, B7-1 and B7-2 surface molecules, lymphocyte receptors, leukocyte receptors, antigen presenting molecules, and the like. Examples of suitable macrophage surface target proteins include, but are not limited to, heparin sulfate proteoglycans, αᵥβ₃ integrins, αᵥβ₅ integrins, B7-1, B7-2, CD11c, CD13, CD16, CD163, CD1a, CD22, CD23, CD29,Cd32, CD33, CD36, CD44, CD45, CD49e, CD52, CD53, CD54, CD71, CD87, CD9, CD98, Ig receptors, Fc receptor proteins (e.g., subtypes of Fcα, Fcγ, Fcε, etc.), folate receptor b, HLA Class I, Sialoadhesin, siglec-5, and the toll-like receptor-2 (TLR2).

When B-cells are the desired target, the gene transfer vector or the conjugate, by way of the amino acid sequence of a subgroup C adenovirus fiber shaft region, recognizes a protein typically found on B-cell surfaces, such as integrins and other adhesion molecules, complement receptors, interleukin receptors, phagocyte receptors, immunoglobulin receptors, activation markers, transferrin receptors, members of the scavenger receptor cysteine-rich (SRCR) superfamily, growth factor receptors, selectins, MHC molecules, TNF-receptors, and TNF-R associated factors. Examples of typical B-cell surface proteins include β-glycan, B cell antigen receptor (BAC), B7-2, B-cell receptor (BCR), C3d receptor, CD1, CD18, CD19, CD20, CD21, CD22, CD23, CD35, CD40, CD5, CD6, CD69, CD69, CD71, CD79a/CD79b dimer, CD95, endoglin, Fas antigen, human Ig receptors, Fc receptor proteins (e.g., subtypes of Fca, Fcg, Fcε, etc.), IgM, gp200-MR6, Growth Hormone Receptor (GH-R), ICAM-1, ILT2, CD85, MHC class I and II molecules, transforming growth factor receptor (TGF-R), α₄β₇ integrin, and αᵥβ₃ integrin.

In accordance with the inventive method, the protein or non-proteinaceous molecule can be delivered to a tumor cell. When tumor cells are the desired target, the gene transfer vector or the conjugate, by way of the amino acid sequence of a subgroup C adenovirus fiber shaft region, recognizes a protein typically found on tumor cell surfaces. The tumor cell can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid). The tumor cell can be associated with cancers of the oral cavity including, for example, the tongue and tissues of the mouth. The tumor cell can be associated with cancers of the digestive system including, for example, the esophagus, stomach, small intestine, colon, rectum, anus, liver, gall bladder, and pancreas. The tumor cell can be associated with cancers of the respiratory system, including, for example, the larynx, lung, and bronchus (e.g., non-small cell lung carcinoma). The tumor cell can be associated with cancers of the uterine cervix, uterine corpus, ovary vulva, vagina, prostate, testis, and penis, which make up the male and female genital systems, and the urinary bladder, kidney, renal pelvis, and ureter, which comprise the urinary system. The tumor cell also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like).

The gene transfer vector and/or the conjugate can contact a particular cell in vivo or ex vivo using methods known in the art. In this respect, the gene transfer vector and/or the conjugate can be administered with a pharmaceutically acceptable carrier and can be delivered to the mammal's cells, preferably human cells, in vivo and/or ex vivo by a variety of mechanisms well-known in the art, such as those described herein. The gene transfer vector and/or the conjugate preferably contacts one or more cells in vivo.

The following examples further illustrate the invention but, of course, should not be construed as in any way limiting its scope.

### EXAMPLE 1

This example demonstrates the ability of an adenovirus ablated for native host cell binding to induce an immune response in a mammal and to transduce immune cells.

A panel of adenovirus vectors was constructed to test the contribution of the shaft region of the adenovirus serotype 5 (Ad5) fiber protein to immunogenicity. Ad5 vectors that expressed the green fluorescent protein (GFP) were modified in the penton base and fiber proteins. Specifically, an integrin binding motif, Arg-Gly-Asp (RGD) was deleted from the penton base protein, and an amino acid residue of the knob region of the fiber protein that mediates interaction of the fiber with the Cocksaxie B and Adenovirus Receptor (CAR) also was deleted. This “double-ablated” Ad5 vector was referred as Adf.DA.

An adenovirus serotype 35 (Ad35) vector was modified such that the shaft region of the Ad35 fiber protein was replaced with the shaft region of the Ad5 fiber protein using routine recombinant DNA techniques. This Ad35 vector, referred to as Ad35f.5S, also encoded the GFP protein. GFP-expressing, wild type capsid vectors of adenovirus serotypes 35 and 5, referred to as Ad35f and Adf, respectively, served as controls.

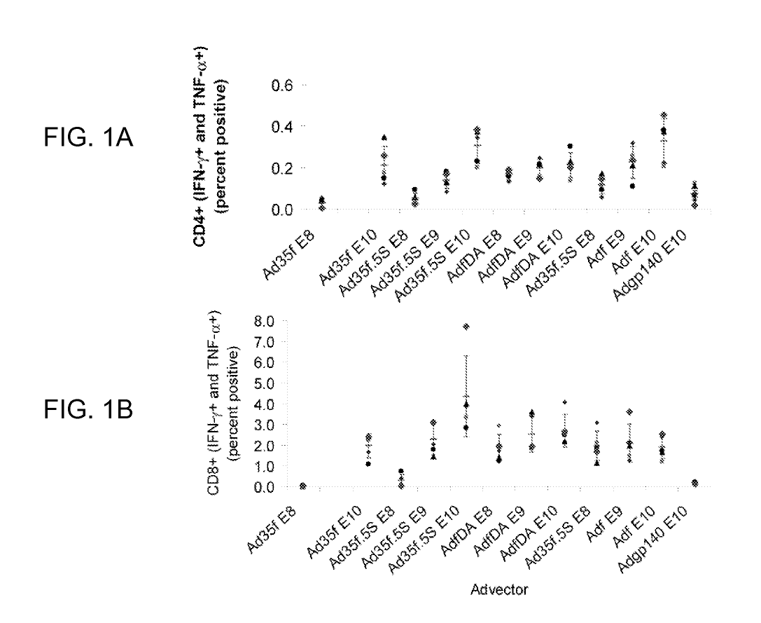

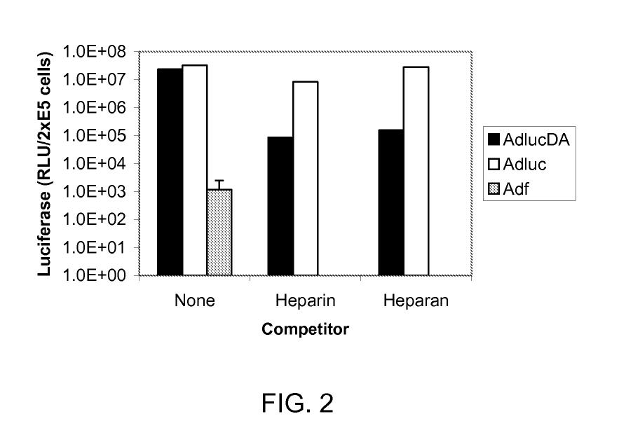

Groups of five Balb/c mice were immunized with the above-described adenoviral vectors over a dose range of 1×10⁸ to 1×10¹⁰ particles (pu). The percentage of T-cells that were immunoreactive to the GFP antigen was determined by intra-cellular cytokine staining (see FIGS. 1A and 1B). Animals immunized with Ad35f.5S exhibited the highest percentage of cytokine positive CD8+ T cells, and exhibited the same level of CD4+ T cells as the control Adf vector. Ad35f.5S induced levels of CD8+ T cells that significantly exceeded the levels induced by Ad35f. The animals immunized with AdfDA also mounted a significant immune response to GFP, and the levels of CD4+ and CD8+ T cells were at least equal to those animals immunized with Adf.

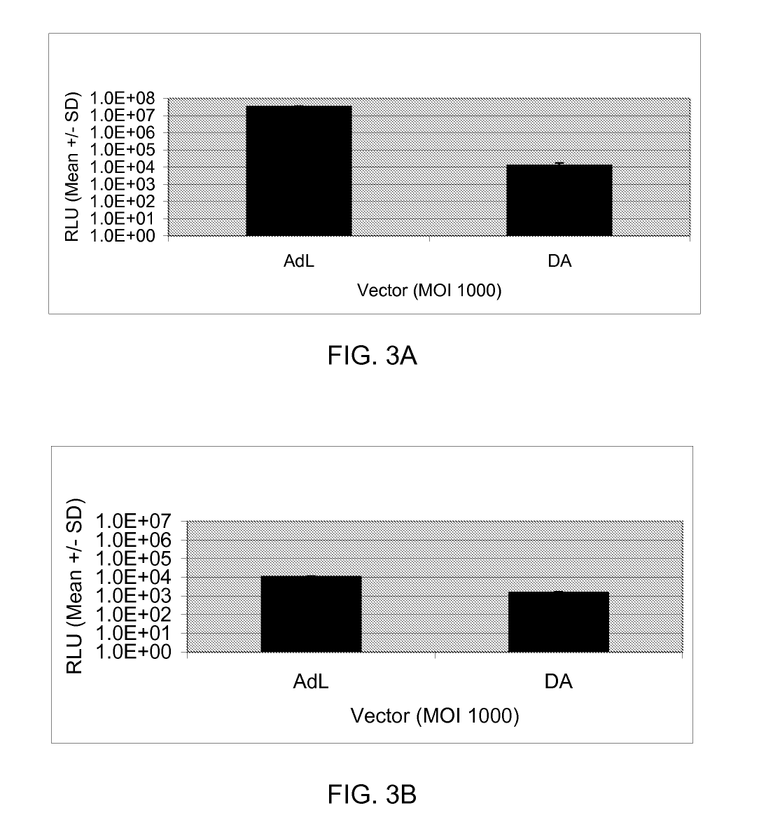

The ability of the double-ablated vector, and the role of the Ad5 shaft region, in transducing cells of the immune system were determined. Murine bone marrow cells were cultured for 1 week with GMCSF, infected with an E1-deficient Ad5 encoding a luciferase reporter gene (AdL), AdLDA, or Adf in either the absence or presence of inhibitors of interaction with heparan sulfate proteoglycans. Competitors were heparin (Sigma Aldrich H6279) or heparan (heparan sulfate proteoglycan, Sigma Aldrich H4777). The population of cells was enriched for dendritic cells by sorting with flow cytometry on CD19 and CD11c. This population of cells is referred to as myeloid dendritic cells (see, e.g., Inaba et al., J Exp. Med., 176, 1693-1702 (1992)). In the absence of competitor, the luciferase activity of CD19-negative, CD11c-positive cells was equivalent between populations infected by AdLDA or AdL (see FIG. 2, AdlucDA and Adluc, respectively). In the presence of competitor, the level of luciferase activity from cells infected with AdLDA was approximately 100-fold lower, showing the dependency of transduction on interaction with heparan sulfate proteoglycan. Since the KKTK motif in the shaft region of Ad5 is known to be a heparan sulfate proteoglycan binding motif (see, e.g., Hileman et al., supra, and Smith et al., supra), these results demonstrate that the double-ablated vector transduces professional antigen presenting cells through a fiber shaft dependent mechanism.

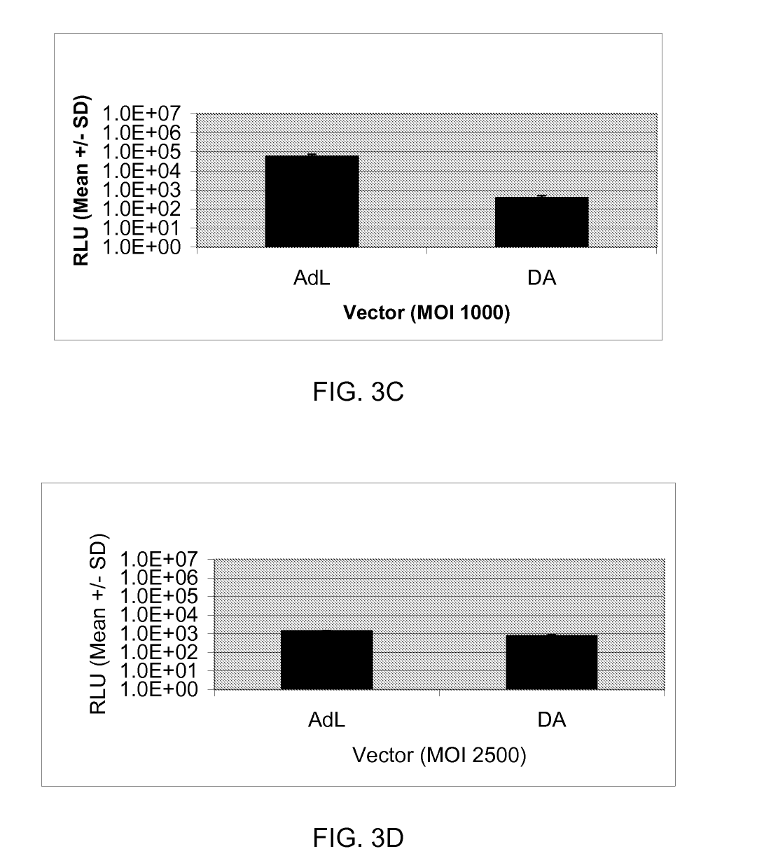

The double-ablated vector was next tested for transduction of tumor cells. The double-ablated vector showed differential transduction relative to the wild type capsid vector. AdL.DA transduction of 293-ORF6 cells resulted in 1000-fold less luciferase activity compared to AdL, whereas the luciferase activity in Caov3 cells, a human adenocarcinoma cell line, was equivalent between AdL and AdL.DA (see FIG. 3A). Similar results were obtained with two murine cell lines (see FIG. 3B).

This example demonstrates that the immune response induced by Ad5 and Ad35 vectors is independent of interactions mediated by penton base protein and the CAR-binding region of fiber protein. Additionally, this example demonstrates that the shaft region of the Ad5 fiber confers specificity for transducing cells of the immune system (e.g., myeloid dendritic cells), as well as tumor cells (e.g., human ovarian adenocarcinoma).

### EXAMPLE 2

This example demonstrates the preparation of the inventive gene transfer vector.

An adeno-associated virus vector is prepared in accordance with the methods disclosed in Warrington et al., J. Virol., 78, 6595-6609 (2004). In particular, an amino acid sequence comprising the amino acid sequence KKTK of the shaft region of an adenovirus serotype 5 fiber protein is inserted at amino acid position 138 within the overlap region of the AAV V1 and V2 capsid proteins. The resulting AAV vector is further engineered to contain an antigen, such as the GAG antigen of HIV. The AAV vector exhibits enhanced immunogenicity when administered to a human as compared to an unmodified AAV vector.

### EXAMPLE 3

This example demonstrates the preparation of the inventive conjugate.

An amino acid sequence comprising the amino acid sequence KKTK (SEQ ID NO: 1) of the shaft region of an adenovirus serotype 5 fiber protein is conjugated to the N-terminus or C-terminus of a recombinant anthrax protective antigen vaccine or a recombinant anthrax lethal factor vaccine (as reviewed in, e.g., Leppla et al., J. Clin. Invest., 109, 141-144 (2002). The conjugate exhibits enhanced immunogenicity when administered to a human as compared to the unmodified protective antigen vaccine or the lethal factor vaccine.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inducing an immune response in a mammal, which method comprises administering to the mammal a gene transfer vector comprising (a) a nucleic acid sequence encoding at least one antigen which is expressed in the mammal to induce an immune response, and (b) an amino acid sequence comprising at least three contiguous amino acids of a shaft region of a subgroup C adenovirus fiber protein, wherein the gene transfer vector is not an adenoviral vector.

2. The method of claim 1, wherein the gene transfer vector is a viral vector.

3. The method of claim 2, wherein the viral vector is selected from the group consisting of adeno-associated virus vectors, retroviral vectors, herpes simplex virus (HSV) vectors, poxvirus vectors, lentiviral vectors, parvovirus vectors, and bacteriophage vectors.

4. The method of claim 1, wherein the gene transfer vector is a liposome, a plasmid, or a virus-like particle.

5. A method of inducing an immune response in a mammal, which method comprises administering to the mammal a conjugate comprising (a) at least one antigen which induces an immune response in the mammal, and (b) an amino acid sequence comprising at least three contiguous amino acids of a shaft region of a subgroup C adenovirus fiber protein, wherein the conjugate is not an adenovirus.

6. The method of claim 5, wherein the conjugate is a fusion protein.

7. The method of claim 1, wherein the amino acid sequence comprises at least three contiguous amino acids of a shaft region of a serotype 5 or serotype 2 adenovirus fiber protein.

8. The method of claim 1, wherein the amino acid sequence comprises the amino acid residues lysine-lysine-threonine-lysine (KKTK) (SEQ ID NO: 1).

9. The method of claim 1, wherein the antigen is selected from the group consisting of a peptide, a protein, or a glycoprotein.

10. The method of claim 1, wherein at least one antigen is selected from the group consisting of env, gag, and pol from clades A, B, or C of a human immunodeficiency virus (HIV), and a fusion protein comprising any of the foregoing.

11. The method of claim 1, wherein at least one antigen is selected from the group consisting of an E protein, an M protein, and a spike protein of a severe acute respiratory syndrome (SARS) virus.

12. The method of claim 1, wherein at least one antigen is isolated from Plasmodium falciparum, Plasmodium vivax, Plasmodium ovale, or Plasmodium malariae.

13. The method of claim 1, wherein at least one antigen is selected from the group consisting of a Dengue protein M, Dengue protein E, Dengue D1NS1, Dengue D1NS2, and Dengue D1NS3.

14. The method of claim 1, wherein the mammal is a human.

15. A gene transfer vector comprising (a) a nucleic acid sequence encoding a protein, and (b) an amino acid sequence comprising at least three contiguous amino acids of a shaft region of a subgroup C adenovirus fiber protein, wherein the gene transfer vector is not an adenoviral vector.

16. The gene transfer vector of claim 15, wherein the nucleic acid sequence encodes an antigen or a cytotoxic protein.

17. The gene transfer vector of claim 15, wherein the gene transfer vector is a viral vector.

18. The gene transfer vector of claim 17, wherein the viral vector is selected from the group consisting of adeno-associated virus vectors, retroviral vectors, herpes simplex virus (HSV) vectors, poxvirus vectors, lentiviral vectors, parvovirus vectors, and bacteriophage vectors.

19. The gene transfer vector of claim 15, wherein the gene transfer vector is a liposome, a plasmid, or a virus-like particle.

20. The gene transfer vector of claim 15, wherein the amino acid sequence comprises at least three contiguous amino acids of a shaft region of a serotype 2 or serotype 5 adenovirus fiber protein.

21. The gene transfer vector of claim 15, wherein the amino acid sequence comprises the amino acid residues lysine-lysine-threonine-lysine (KKTK) (SEQ ID NO: 1).

22. A conjugate comprising (a) a protein or a non-proteinaceous molecule, and (b) an amino acid sequence comprising at least three contiguous amino acids of a shaft region of a subgroup C adenovirus fiber protein, wherein when the conjugate comprises a protein, the conjugate is not an adenovirus.

23. The conjugate of claim 22, wherein the conjugate comprises a protein.

24. The conjugate of claim 23, wherein the protein is an antigen or a cytotoxic protein.

25. The conjugate of claim 22, wherein the conjugate is a fusion protein.

26. The conjugate of claim 22, wherein the conjugate comprises a non-proteinaceous molecule.

27. The conjugate of claim 26, wherein the non-proteinaceous material is a small molecule.

28. The conjugate of claim 27, wherein the small molecule is a hapten.

29. The conjugate of claim 22, wherein the amino acid sequence comprises at least three contiguous amino acids of a shaft region of a serotype 2 or serotype 5 adenovirus fiber protein.

30. The conjugate of claim 22, wherein the amino acid sequence comprises the amino acid residues lysine-lysine-threonine-lysine (KKTK) (SEQ ID NO: 1).

31. A method of delivering a protein to a cell, which method comprises contacting the cell with the gene transfer vector of claim 15, whereby the nucleic acid sequence is expressed and the protein is produced.

32. A method of delivering a protein to a cell, which method comprises contacting the cell with the conjugate of claim 23, whereby the nucleic acid sequence is expressed and the protein is produced.

33. A method of delivering a non-proteinaceous molecule to a cell, which method comprises contacting the cell with the conjugate of claim 26.

34. The method of claim 31, wherein the cell is a dendritic cell.

35. The method of claim 31, wherein the cell is a tumor cell.


SEQUENCE LISTING (3 sequences)

SEQ ID NO: 1 - protein, 4 aa, Adenovirus
KKTK

SEQ ID NO: 2 - protein, 3 aa, Adenovirus
KKT

SEQ ID NO: 3 - protein, 3 aa, Adenovirus
KTK